(19) United States
(12) Patent Application Publication
(10) Pub. No.: US 2009/0030296 A1
(43) Pub. Date: Jan. 29, 2009

(54) Predictive oximetry model and method

(76) Inventor: Bernhard B. Sterling, Danville, CA (US)

Correspondence Address: Francis Law Group, 1942 Embarcadero, Oakland, CA, 94606, US

(21) Appl. No.: 11/881,103

(22) Filed: Jul. 24, 2007

(51) Int. Cl. A61B 5/1455

(52) U.S. Cl. 600/323; 600/331

(57) ABSTRACT

The invention comprises a method for determining oxygen saturation in a subject, comprising the steps of compiling a data base of measured spectral data that includes pulsatile AC and non-pulsatile DC components, and spectral values of oxyhemoglobin (oxyHb) and deoxyhemoglobin (deoxyHb); determining absorbed pulsatile components and non-pulsatile components as a function of the oxyHb and deoxyHb values; determining total pulsatile and non-pulsatile optical density as a function of the absorbed pulsatile and non-pulsatile components; determining a mathematical relationship between at least one pulsatile AC parameter and at least one non-pulsatile DC parameter; and estimating oxygen saturation based on the mathematical relationship.

### FIELD OF THE PRESENT INVENTION

The present invention relates to the field of pulse oximetry. More specifically, the invention relates to a mathematical model and method for predicting physical and physiological characteristics based on spectral pulse oximetry data.

### BACKGROUND OF THE INVENTION

It is well known in the art that pulse oximetry is based on the principle that the color of blood is related to the oxygen saturation level of hemoglobin. Indeed, as blood deoxygenates, the pinkish skin color (in many individuals) transitions to a bluish hue. This phenomenon allows measurements of the degree of oxygen saturation of blood using, what is commonly referred to as, optical pulse oximetry technology.

Pulse oximetry devices, i.e. oximeters, typically measure and display various blood constituents and blood flow characteristics including, blood oxygen saturation of hemoglobin in arterial blood, the volume of individual blood pulsations supplying the flesh and the rate of blood pulsations corresponding to each heartbeat of the patient (see FIG. 1, discussed in detail herein). Illustrative are the devices disclosed in U.S. Pat. Nos. 5,193,543; 5,448,991; 4,407,290; and 3,704,706.

As is well known in the art, a pulse oximeter passes light through human or animal body tissue where blood perfuses the tissue, such as a finger or ear, and photoelectrically senses the absorption of light in the tissue. Since oxygenated and deoxygenated hemoglobin absorb visible and near infrared light differently, two lights having discrete frequencies in the range of about 650-670 nm in the red range and about 800-1000 nm in the infrared range are typically passed through the tissue. The amount of transmitted light passed through the tissue varies in accordance with the changing amount of blood constituent, i.e. oxygen (or oxygen saturation), in the tissue and the related light absorption.

Two oxygen saturation parameters can readily be ascertained via oximetry: arterial oxygen saturation (SaO₂) is based on direct measurement of light absorption in tissue and/or blood based on all commonly measured hemoglobin components. Peripheral, arterial oxygen saturation (SpO₂), as measured by pulse oximetry, is generally determined by measuring the constant (non-pulsatile) and pulsatile light intensities (discussed below) of the two functional components oxyhemoglobin and deoxyhemoglobin hemoglobin, at each of the two noted wavelengths, and correlating the measured intensities to provide peripheral oxygen saturation.

Light absorption measured via pulse oximetry typically includes a constant (non-pulsatile) component and a variable (pulsatile) component. The constant component is commonly referred to as the “DC” component. The DC component is influenced by several factors, such as the light absorbency of the biological tissue, presence of venous blood, capillary blood and non-pulsatile arterial blood, light scattering properties of tissue, intensity of the light source and sensitivity of the detector.

The variable (pulsatile) component is commonly referred to as the “AC” component. The variable component results from the pulsatile flow of arterial blood through the tissue being probed—light absorption varies proportionately to the flow of blood. Thus, since the pulsatile flow is a function of the fluctuating volume of arterial blood, the AC light intensity level closely reflects the light absorption of the oxygenated and deoxygenated hemoglobin of arterial blood.

As is well known in the art, the ratio (R) of pulsatile light intensities to non-pulsatile light intensities is commonly employed to determine peripherally and non-invasively the arterial oxygen saturation (SpO₂), To determine the ratio (R) of pulsatile light intensities to non-pulsatile light intensities, the constant DC component of the light intensity must be factored out. Since the amplitudes of both the AC and DC components are dependent on the incident light intensity, dividing the AC level by the DC level provides a “corrected” AC level that is no longer a function of the incident light intensity, i.e. AC/DC. Ratio (R) can thus be derived as follows:

R=(AC₁/DC₁)/(AC₂/DC₂) (Eq. 1)

Ratio (R) is a well accepted representation or indicator of arterial oxygen saturation, i.e. SaO₂. An empirically derived calibration curve for the relationship between ratio R and SaO₂ is then typically employed to determine the functional oxygen saturation, i.e. SpO₂.

The measured transmission of light transmitted through blood-perfused tissue and the oxygen saturation level (SpO₂) determined therefrom are therefore based on two primary factors: (i) the natural difference in light absorption in oxygenated hemoglobin and deoxygenated hemoglobin and (ii) the detected change in light absorption resulting from the fluctuating volume of arterial blood passing through the tissue between the light source and the sensor, i.e. the pulsatile component.

The amplitude of the pulsatile component is, however, typically a small fraction of the total signal amplitude. Thus, small changes in the pulsatile component can, and in many instances will, be “lost” in the background of the total signal amplitude.

As is well known in the art, the light(s) transmitted to biological tissues will, in most instances, scatter and be absorbed by the tissue being probed. Light scattering, i.e. background scattering, and absorption can, and in many instances will, have a significant impact on oximeter accuracy. See, e.g., Fine, et al., “Multiple-Scattering Effects in Transmission Oximetry”, Medical and Biological Engineering & Computing, vol. 31(5), pp. 516-522 (September 1993).

As is also well known in the art, conventional pulse oximeters and methodologies rely on the pulsatile component. Further, conventional pulse oximeters and methodologies do not, and cannot, effectively account for light scattering and absorption of light in the biological tissues that are being probed. Thus, conventional methodologies (or techniques) typically employ empirical data and factor in an average component for scattering and absorption. See e.g., De Kock, et al., “Pulse Oximetry: Theoretical and Experimental Models”, Medical and Biological Engineering & Computing, vol. 31 (1993). This approach results in pulse oximeters that rely upon fixed calibration curves to predict SpO₂ from measured electronic signals.

The current practice in pulse oximetry of subsuming the scattering and absorption of light that occurs in tissue by resorting to empirical calibration techniques is problematic. While it may be acceptable at oxygen saturation levels within normal ranges for adults, i.e. 70% to 100% SaO₂, it becomes less acceptable when oxygen saturation is in the lower range, e.g., 15% to 65% SaO₂, which is commonly encountered in fetal oximetry and severe hypoxia in post-natal subjects.

The size of the pulse oximeter probe can also adversely affect oxygen saturation determination. In oximeters with larger probes, e.g., probes having a path length between the emitter and detector that would encompass a finger, foot or earlobe, the conventional calibration approach is acceptable because scatter and absorption are less of an issue. However, as the probe size decreases and the path length becomes shorter, e.g., fetal oximeter probes having a path length less than 5 mm, the error due to background scattering and absorption has a relatively greater impact on oximeter accuracy.

Various techniques have thus been employed to account for the effects of light scattering and absorption in measured AC and DC signals. Illustrative are the techniques described in Marble et al., “Diffusion-based Model Pulse Oximetry: In vitro and In vivo Comparisons”, Applied Optics, vol. 33, no. 7 (1994) and De Kock, et al., “Pulse Oximetry: Theoretical and Experimental Models”, Medical and Biological Engineering & Computing, vol. 31 (1993), wherein the scattering and absorption characteristics of the probed biological tissue are theoretically modeled.

A major drawback associated with the theoretical approach disclosed in the noted references is that the number of variables used in the various models makes it difficult to accurately model the scattering and absorption characteristics of the probed tissue. This results in further approximations, and in an inevitable “guessing” of some of the parameters. For example, in order to calculate absorption from the DC signal, one needs to “guess” the scope and/or effect of light scattering. Similarly, where one desires to determine light scattering from the DC signal, absorption needs to be approximated.

In U.S. Pat. No. 6,839,580, a further theoretical technique is disclosed, wherein clinical data is used to determine an average scattering. The average scattering value is incorporated into a parameter, i.e. multiplier, identified as kDc, which is employed in a “calibration equation” with ratio (R) to determine a SpO₂ value.

Although the technique disclosed in the '580 patent provides an effective means for calibrating a pulse oximeter device, the technique is similarly limited in its effectiveness and, hence, utility. For example, the technique does not account for the effects of venous pulsation or variations in tissue thickness or density.

It would therefore be desirable to provide an improved oximetry method and model that effectively and accurately accounts for the effects of scattering and absorption in measured AC and DC pulse oximetry signals.

It is therefore an object of the present invention to provide an improved oximetry model and method for determining SaO₂ and SpO₂ that substantially reduces or eliminates the disadvantages and drawbacks associated with conventional oximetry models and methodologies.

It is another object of the invention to provide an oximetry model and method that can provide an estimate of oxygen saturation based on a mathematical relationship between a pulsatile AC parameter and a DC non-pulsatile parameter.

It is another object of the invention to provide an oximetry model and method that provides a pulse amplitude corrected ratio of logarithms that can be employed to provide a more accurate determination of SaO₂ and SpO₂.

It is another object of the invention to provide an oximetry model and method that can predict the effects of multiple physiological parameters, including, without limitation, venous pulsation, scattering pulsation, and biological tissue thickness, density and perfusion, and variations thereof, and provides effective means therefore.

### SUMMARY OF THE INVENTION

In accordance with the above objects and those that will be mentioned and will become apparent below, in one embodiment of the invention, there is provided an oximetry model and method for predicting oxygen saturation, comprising (i) compiling a data base of measured spectral data characterizing the transmission of light through a tissue of interest in the subject, the spectral data including pulsatile AC and non-pulsatile DC components of the transmitted light, and spectral values of oxyhemoglobin (oxyHb) and deoxyhemoglobin (deoxyHb), (ii) determining light scattering intensity (I) of the AC and DC components, (iii) determining absorbed pulsatile components and non-pulsatile components as a function of the oxyHb and deoxyHb values, (iv) determining total pulsatile optical density as a function of the absorbed pulsatile components and the AC component scattered light intensity, (v) determining total non-pulsatile optical density as a function of the absorbed non-pulsatile components and the DC component scattered light intensity, (vi) determining a mathematical relationship between at least one pulsatile AC parameter and at least one non-pulsatile DC parameter, and (vii) estimating oxygen saturation based on the mathematical relationship.

In one embodiment of the invention, the pulsatile AC parameter comprises a total pulsatile transmittance equivalent value and the non-pulsatile DC parameter comprises a total non-pulsatile transmittance equivalent value.

In one embodiment, the method includes the steps of determining the total pulsatile transmittance equivalent value as a function of the total pulsatile optical density and determining the total non-pulsatile transmittance equivalent value as a function of the total non-pulsatile optical density.

In another embodiment of the invention, the oximetry model and method for predicting oxygen saturation comprises (i) compiling a data base of measured spectral data characterizing the transmission of light through a tissue of interest in the subject, the spectral data including pulsatile AC and non-pulsatile DC components of the transmitted light, and spectral values of oxyhemoglobin (oxyHb) and deoxyhemoglobin (deoxyHb), (ii) determining light scattering intensity of the AC and DC components,

(iii) determining absorbed pulsatile components and non-pulsatile components as a function of the oxyHb and deoxyHb values, (iv) determining total pulsatile optical density as a function of the absorbed pulsatile components and the AC component scattered light intensity, (v) determining total non-pulsatile optical density as a function of the absorbed non-pulsatile components and the DC component scattered light intensity, (vi) determining at least one total pulsatile transmittance equivalent value as a function of the total pulsatile optical density, (vii) determining at least one total non-pulsatile transmittance equivalent value as a function of the total non-pulsatile optical density, (viii) determining a ratio of logarithms as a function of the pulsatile and non-pulsatile transmittance equivalent values, and (ix) estimating oxygen saturation based on the ratio of logarithms.

In another embodiment of the invention, the oximetry model and method for predicting oxygen saturation comprises (i) providing a pulse oximetry system, the pulse oximetry system including a tissue probe having a radiation emitter that is adapted to transmit light having a first wavelength through a tissue of interest and a radiation detector that is adapted to receive the transmitted light after transmission through the tissue, (ii) measuring the transmission of light through the tissue of interest in the subject with the pulse oximetry system, (iii) compiling a data base of measured spectral data characterizing the transmission of light through the tissue of interest, the spectral data including pulsatile AC and non-pulsatile DC components of the transmitted light, and spectral values of oxyhemoglobin (oxyHb) and deoxyhemoglobin (deoxyHb), (iv) determining light scattering intensity of the AC and DC components, (v) determining light scattering intensity of the AC and DC components, (vi) determining absorbed pulsatile components and non-pulsatile components as a function of the oxyHb and deoxyHb values, (vii) determining total pulsatile optical density as a function of the absorbed pulsatile components and the AC component scattered light intensity, (viii) determining total non-pulsatile optical density as a function of the absorbed non-pulsatile components and the DC component scattered light intensity, (ix) determining at least one total pulsatile transmittance equivalent value as a function of the total pulsatile optical density, (x) determining at least one total non-pulsatile transmittance equivalent value as a function of the total non-pulsatile optical density, (xi) determining a ratio of logarithms as a function of the pulsatile and non-pulsatile transmittance equivalent values, and (xii) estimating oxygen saturation based on the ratio of logarithms.

In accordance with another embodiment of the invention, there is provided a method of calibrating a pulse oximetery system comprising (i) providing a pulse oximetry system, the pulse oximetry system including a tissue probe having a radiation emitter that is adapted to transmit light having a first wavelength through a tissue of interest and a radiation detector that is adapted to receive the transmitted light after transmission through the tissue, (ii) measuring the transmission of light through the tissue of interest in the subject with the pulse oximetry system, (iii) compiling a data base of measured spectral data characterizing the transmission of light through a tissue of interest in the subject, the spectral data including pulsatile AC and non-pulsatile DC components of the transmitted light, and spectral values of oxyhemoglobin (oxyHb) and deoxyhemoglobin (deoxyHb), (iv) determining light scattering intensity of the AC and DC components, (v) determining absorbed pulsatile components and non-pulsatile components as a function of the oxyHb and deoxyHb values, and (vi) determining a corrected ratio of logarithms based on the absorbed pulsatile and non-pulsatile components, and the AC and DC component scattered light intensities, the corrected ratio of logarithms representing oxygen saturation in a subject.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a schematic illustration of a plethysmographic waveform and the related r-wave portion of an electrocardiogram waveform;

FIG. 2 is a schematic illustration of a pulse oximeter apparatus, according to the invention;

FIG. 3 is a graphical illustration of optical density (OD) spectra at relevant wavelengths for oximetry of base absorbances (DC) of oxygenated and de-oxygenated blood, and the wavelength-dependent magnitude of light scattering in relationship to absorbance, according to the invention;

FIG. 4 is a graphical illustration of OD spectra at relevant wavelengths for oximetry of variable absorbances (AC), an example at one condition of oxygenated and de-oxygenated blood and the wavelength-dependent magnitude of light scattering in relationship to absorbance, according to the invention;

FIG. 5 is a graphical illustration of the ratio of logarithms below 70% O₂ saturation based on a data fit from 70-100% saturation, according to the invention;

FIG. 6 is a graphical illustration of the effects of pulse amplitude on the ratio of logarithms over a range of O₂ saturations, according to the invention;

FIG. 7 is a graphical illustration of the effects of percent venous pulsatile component of total pulse amplitude on the ratio of logarithms over a range of O₂ saturations, according to the invention;

FIG. 8 is a graphical illustration of the effects of percent non-optically absorbing pulsatile component of total pulse amplitude on the ratio of logarithms over a range of O₂ saturations, according to the invention;

FIG. 9 is a graphical illustration of the effects of variations in relative finger thickness on the ratio of logarithms over a range of O₂ saturations, according to the invention;

FIG. 10 is a graphical illustration of the effects of variations in the density of biological tissue on the ratio of logarithms over a range of O₂ saturations, according to the invention; and

FIG. 11 is a graphical illustration of the effects of variations in relative finger perfusion on the ratio of logarithms over a range of O₂ saturations, according to the invention.

### DETAILED DESCRIPTION OF THE INVENTION

Before describing the present invention in detail, it is to be understood that this invention is not limited to particularly exemplified methods, systems or circuitry as such may, of course, vary. Thus, although a number of methods and systems similar or equivalent to those described herein can be used in the practice of the present invention, the preferred methods and systems are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise.

### Definitions

The term “signal”, as used herein, is meant to mean and include an analog electrical waveform or a digital representation thereof, which is collected from a biological or physiological sensor.

The term “optical density”, as used herein, and means and includes, without limitation, absorbance of light by a medium, as defined and quantified by Beer's Law, as well as practically measured in turbid media, such as human tissue.

The terms “O₂ saturation” and oxygen saturation”, as used herein, and mean and include, without limitation, arterial oxygen saturation, i.e. SaO₂, and functional (and/or peripheral) oxygen saturation, i.e. SPO₂.

The terms “patient” and “subject”, as used herein, is meant to mean and include humans and animals.

The present invention substantially reduces or eliminates the disadvantages and drawbacks associated with conventional oximetry models (and/or algorithms) and methodologies. As discussed in detail herein, a semi-empirical model is provided, which can be effectively employed for predicting physical and physiological characteristics based on spectral pulse oximetry data.

The present invention differs from conventional methodologies in that, in one embodiment, the model is based on “clinical” spectral data of oxy- and deoxy-hemoglobin and an empirical expression, i.e. mathematical equation, for light scattering.

As set forth in detail herein, the model and method employing same have been validated by accurately predicting ratios of logarithms as functions of pulse amplitudes and saturations. As will be appreciated by one having skill in the art, such predictions are useful for the purpose of indexing each ratio of logarithms to a nearest tabulated value, whereby, a pulse amplitude corrected ratio of logarithms is obtained for a more accurate determination of oxygen saturation. Another example of an important utility of the oximetry model of the invention is the prediction of the ratio of logarithms at low saturations, i.e. saturations below 70%, which are not accessible by experimentation on human subjects.

The oximetry model and method are also useful for predicting (i) the effects of venous pulsation, (ii) the effects of non-optically absorbing pulsatile component and (iii) any instrument and/or sensor characteristic, such as light emission and transmission characteristics, as a function of optical cuvette, e.g. a human finger thickness, emitter and detector characteristics in combinations, and providing effective corrective means therefore. Corrective means provided by the oximetry model and method can also be applied to data based on the measured DC level.

As stated above, pulse oximeters typically provide at least two outputs; a first output reflecting the blood oxygen saturation of hemoglobin in arterial blood and a second output reflecting the rate of blood pulsations corresponding to each heartbeat of the patient, which is commonly represented by a “plethysmographic wave or waveform”.

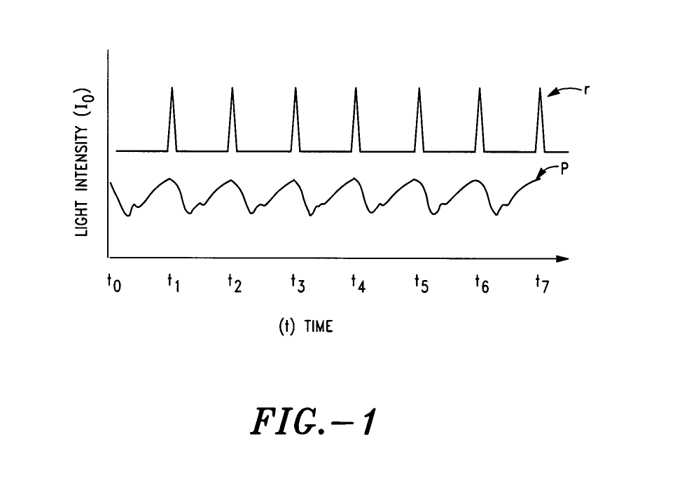

Referring now to FIG. 1, there is shown a graphical illustration of a plethysmographic waveform (designated “p”) and the related “r-wave” portion of an electrocardiogram (ECG) waveform (designated “r”).

As is well known in the art, an ECG waveform comprises a complex waveform having several components that correspond to electrical heart activity. A key component is the QRS component, which relates to ventricular heart contraction. The r-wave portion of the QRS component is typically the steepest wave therein, having the largest amplitude and slope, and is typically deemed an accurate indication of the onset of cardiovascular activity.

As illustrated in FIG. 1, the flow of arterial blood typically follows the r-wave of the electrical heart activity by a determinable period of time that remains essentially constant for a given patient. See, e.g., Goodlin et al., Systolic Time Intervals in the Fetus and Neonate, Obstetrics and Gynecology, Vol. 39, No. 2, (February 1972) and U.S. Pat. No. 3,734,086.

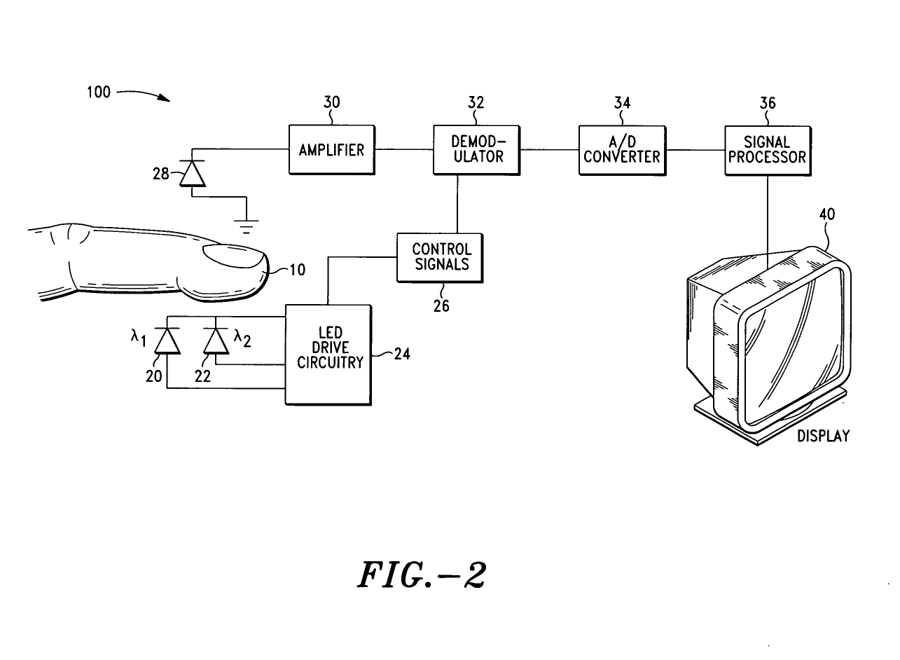

Referring now to FIG. 2, there is shown one embodiment of a pulse oximeter and associated system (denoted generally “100”) that can be employed within the scope of the present invention. As illustrated in FIG. 2, the system 100 preferably includes two emitters 20, 22 and detector 28, which are positioned adjacent the tissue being analyzed, i.e. finger 10.

Two lights are emitted by the emitters 20, 22; in one embodiment, a first light having a discrete wavelength in the range of approximately 650-670 nanometers in the red range and a second light having a discrete wavelength in the range of approximately 800-950 nanometers. The lights, in the illustrated embodiment, are transmitted through finger 10 via emitters 20, 22 and detected by detector 28.

The emitters 20, 22 are driven by drive circuitry 24, which is, in turn, governed by control signal circuitry 26. Detector 28 is in communication with or connected to amplifier 30. The signal from amplifier 30 is transmitted to demodulator 32, which is also synchronized to control signal circuitry 24. The demodulator 32, which is employed in most pulse oximeter systems, removes any common mode signals present and splits the time multiplexed signal into two (2) channels, one representing the red voltage (or optical) signal and the other representing the infrared voltage (or optical) signal.

The signal from the demodulator 32 is transmitted to an analog-digital converter 34. As is well known in the art, the output signal from the demodulator 34 is typically a time multiplexed signal comprising (i) a background signal, (ii) the red light range signal, and (iii) the infrared light range signal.

The desired computations are performed on the output from the converter 34 by signal processor 36 and the results transmitted to and displayed by display 40.

As indicated above, in a preferred embodiment of the invention, the predictive model and method of the invention is based on “clinical” spectral data of oxy- and deoxy-hemoglobin and an empirical expression, i.e. mathematical equation, for light scattering. In the embodiment described herein, oximetry reference data for oxyhemoglobin (oxyHb) and deoxyhemoglobin (deoxyHb) were thus obtained from published literature, i.e. S. Prahl, “Tabulated Molar Extinction Coefficient for Hemoglobin in Water” (http://omlc.ogi.edu/spectra/hemoglobin/summary.html). As set forth in detail below, these coefficients were converted to optical densities (OD).

As will be appreciated by one having ordinary skill in the art, spectral values for oxyHb and deoxyHb can also be provided by performing conventional optical blood oximetry on a subject.

Thus, in one embodiment of the invention, measured spectral data characterizing the transmission of light through a tissue of interest in a subject is compiled; the spectral data including pulsatile AC and non-pulsatile DC components of the transmitted light, and spectral values for oxyHb and deoxHb.

A key parameter in the oximetry model (and method) of the invention is the light scattering intensity of the AC and DC components. According to the invention, light scattering intensity of the AC and DC components can be determined by various conventional methodologies and mathematical equations and/or relationships. In one embodiment of the invention, light scattering intensity (“I”) is modeled as mie scattering, i.e.

I=s/(λ^{exp}) (Eq. 2)

where:
λ=wavelength; and
s=an adjustment factor.

According to the invention, factor “s” is obtained from independent experimental data to produce an attenuation by scattering alone that is realistic for the physical body site that's probed, such as the index finger or an ear lobe. In one embodiment of the invention, factor s is adjusted to produce between 60% and 80% of total DC at 805 nm when the total DC, as defined by the sum of OD (oxyHb plus deoxyHb) and attenuation due to scattering at intensity I reaches a level that is typically seen in pulse oximetry, e.g., a total OD of 1.5.

According to the invention, the wavelength exponent is derived by curve fitting experimental “clinical” data of the scattering term(s) of human tissue. As will be appreciated by one having ordinary skill in the art, the exponent will vary as a function of wavelength of the light and the physical measurement site of the person. In one embodiment of the invention, the wavelength exponent (exp) is equal to approximately 1.46.

In a preferred embodiment, the pulsatile components (AC) of the total pulse amplitude and non-pulsatile components (DC) are determined from the fundamental spectral data, as described above. The total pulsatile optical density (“OD”), i.e. OD_{t,p}, is then determined at each wavelength of interest as follows:

OD_{t,p}=(a*OD_{a,p})+(b*OD_{v,p})+(c*OD_{o,p})+(d*I_{s,p}) (Eq. 3)

where:
a=fractional coefficient for arterialized, fully oxygenated, pulsatile optical density;
b=fractional coefficient for venous, fully deoxygenate pulsatile optical density;
c=fractional coefficient for pulsatile optical density of other hemoglobin (Hb) components, such as carboxy- and met-hemoglobin;
d=fractional coefficient for pulsatile scattering intensity;
I_{s,p}=pulsatile scattering intensity;
OD_{a,p}=arterialized, fully oxygenated pulsatile optical density;
OD_{v,p}=venous, fully deoxygenated pulsatile optical density; and
OD_{o,p}=pulsatile optical density of other hemoglobin (Hb) components.

As will be appreciated by one having ordinary skill in the art, all optical density (“OD”) values are effectively larger in the presence of light scattering due to larger effective path length and multiple scattering events. Thus, all OD values may be multiplied by a function of (d*I_{s,p}), which will introduce a small wavelength dependent effect. The fundamental predicted values by the disclosed model are, however, only minimally affected.

As will also be appreciated by one having skill in the art, in pulse oximetry, although the measured “AC” component is equivalent to transmittance (radiation intensity out of the sample under interrogation, as received by the detector divided by radiation intensity in), it is the logarithm of that quantity, i.e. “OD”, which exhibits a linear relationship with concentration and path length. Therefore, as illustrated in Eq. 3 above (and Eq. 4 below), in a preferred embodiment of the invention, the total optical density is represented by the sum of all OD values plus the scattering intensity. The scattering intensity is pragmatically justified to be treated as if it were an absorbance in that at the detector level it is impossible to distinguish lost absorbed photons from lost scattered photons.

According to the invention, total pulsatile OD_{t,p} is preferably always composed of the sum of the functional hemoglobin components at the desired adjustable saturation. By way of example, at 80% saturation in the presence of an additional 2% of “other” Hb components, OD equals the sum of (0.784*(oxyHb−OD))+(0.196*deoxyHb−OD)+(0.02*otherHb−OD)). The “other” hemoglobin (Hb) components can comprise carboxyhemoglobin (carboxyHb) or sickle cell Hb or methemoglobin (metHb). The “other” Hb components can also comprise any intravascular absorbing substance, such as a dye.

According to the invention, to estimate the effects of pulsatile venous blood, the venous components can be calculated at 50% of arterial saturation and 50% of total blood volume, both of which are adjustable, and added to (b*OD_{v,p}) by increasing the fractional coefficient “b” accordingly. The venous saturation can be assumed to be different in different tissues. The venous saturation is also highly variable by cardiac output, perfusion, and oxygen extraction efficiency.

It is also best to experimentally determine venous blood volume by compression exsanguinations. In one embodiment, light scattering at all wavelengths is estimated at sufficient accuracy by fully ex-sanguinating the site above arterial pressure.

According to the invention, fractional coefficients “d” (see Eq. 3 above) and “h” (see Eq. 4, below) are different for different measurement sites and can range from almost 0, e.g., for lyzed blood, to nearly 1 times the total absorbances and scattering for tissue exhibiting very low blood perfusion. In one embodiment, coefficients “d” and “h” are preferably in the range of approximately 0.03-0.1 for “d”, and 0.3-0.8 for “h”, times the total absorbances and scattering.

In one embodiment of the invention, the non-pulsatile (np) components' absorbances, i.e. optical density “OD_{t,np}”, are defined and determined at each wavelength of interest as follows:

OD_{t,np}=(e*OD_{a,np})+(f*OD_{v,np})+(g*OD_{o,np})+(h*I_{s,np}) (Eq. 4)

where:
e=fractional coefficient for arterialized, fully oxygenated non-pulsatile optical density;
f=fractional coefficient for venous, fully deoxygenated non-pulsatile optical density;
g=fractional coefficient for non-pulsatile optical density of other hemoglobin (Hb) components, such as carboxy- and met-hemoglobin;
h=fractional coefficient for non-pulsatile scattering intensity;
I_{s,np}=non-pulsatile scattering intensity;
OD_{a,np}=arterialized, fully oxygenated non-pulsatile optical density;
OD_{v,np}=venous, fully deoxygenated non-pulsatile optical density; and
OD_{o,np}=non-pulsatile optical density of other hemoglobin (Hb) components.

In one embodiment, factors “a” through “h” are introduced as adjustable factors to make the model as versatile as possible and, hence, to fit the actual physical site and the desired patient population. Although it is mathematically required to use OD when quantities are treated as additives, as shown in Eq. 4 above, unless the primary output voltage is modified in hardware or software to provide a mathematical conversion, e.g., log function, the oximeter's voltage output is equivalent to transmittance.

Thus, for quantitative equivalence of the model's output to that of an opto-electronic instrument, wherein the intensity of the radiation sources is adjusted until the DC output voltage of the system is at a pre-set value, e.g., 1 V, in one embodiment, the total transmittance of the calculated DC is multiplied by a factor that results in the model's DC—equivalent transmittance to be approximately 1, which is the sum of all non-pulsatile components' transmittance. As will readily apparent by one having ordinary skill in the art, virtually any other value can be employed.

In order to provide a more robust and realistic model, the calculated transmittances are preferably averaged over the wavelength band of the emitted radiation—the terms AC and DC are borrowed from the field of electrical engineering to illustrate the nature of optical transmitance as a time-variable component caused by the pulsatile nature of blood pressure (AC), as well as the sum of the non-pulsatile base components (DC).

According to the invention, for the estimation of the effects of non-pulsatile venous blood, venous components can be calculated at 50% of arterial saturation and 50% of total blood volume, both of which are adjustable, and added to (f*OD_{v,p}) by increasing the fractional coefficient f accordingly. The venous saturation can thus be assumed to be different in different tissues. The venous saturation is also highly variable by cardiac output, perfusion, and oxygen extraction efficiency.

As will be appreciated by one having ordinary skill in the art, the range of venous blood volume can be substantially less than 50%, e.g., 20%, in a free-flowing site that is above the heart and as high as 80% in a site where venous blood is pooled due to very low venous return. The actual volume of venous blood that is accessible to interrogation by visible and near IR radiation is thus best determined experimentally for a given tissue site.

According to the invention, the combined absorbances, i.e. OD_{t,p} and OD_{t,np}, are then converted to transmittance equivalent values in order to become equivalent to conventional inputs and signal conversions of oximetry instrumentation. As will be appreciated by one having ordinary skill in the art, the combined absorbances can be converted to transmittance equivalent values by various conventional means, including hardware, firm ware and various mathematical algorithms.

In one embodiment of the invention, the total combined absorbances, i.e. OD_{t,p} and OD_{t,np}, are converted to transmittance equivalent values “T” according to the following equation,

T= 1/10^{OD} (Eq. 5)

Thus,

Total pulsatile transmittance (Tₚ)= 1/10^{ODt,p} (Eq. 6)

and

Total non-pulsatile transmittance (T_{n,p})= 1/10^{ODt,np} (Eq. 7)

Preferably, the total pulsatile and non-pulsatile transmittances (Tₚ and T_{n,p}) are determined at all wavelengths of interest.

According to the invention, a mathematical relationship between the total pulsatile and non-pulsatile transmittances is then determined, whereby oxygen saturation can be estimated therefrom. The simplest useful mathematical relationship range is the linear ratio of the pulsatile transmittances at two wavelengths, which is particularly useful at low pulse amplitudes, i.e.

R=T_{p@W1}/T_{p@W2} (Eq. 8)

where:
W1=a first wavelength; and
W2=a second wavelength.

In a preferred embodiment of the invention, the relationship of the pulsatile red and the pulsatile infra-red transmittance (or electronic instrument term “AC”) is preferably defined and determined by, what is commonly referred to as, the “ratio of logarithms R” (or ratio R), i.e.

R=(log(T_{p@W1}/log T_{n,p@W1}))/(log(T_{p@W2}/log T_{n,p@W2})) (Eq. 9)

where:
W1=a first wavelength; and
W2=a second wavelength.

In one embodiment of the invention, W1 is equal to approximately 660 nm and W2 is equal to approximately 920 nm.

As will be apparent to one having ordinary skill in the art, the mathematical relationship log T_{p@W1}/log T_{n,p@W2} is functionally equivalent to the relationship log AC_{@W1}/log DC_{@W2}.

The ratio of logarithms “R”, as determined by Eq. 8 above, effectively and accurately accounts for the effects of scattering and absorption in the measured AC and DC oximetry signal and, hence, provides a more accurate representation of arterial oxygen saturation, i.e. SaO₂. According to the invention, a conventional, empirically derived calibration curve reflecting the relationship between R and SaO₂ can be employed to determine the functional oxygen saturation, i.e. SpO₂.

Referring now to FIG. 3, there is shown a graphical illustration of OD spectra at relevant wavelengths for oximetry of base absorbances (DC) of oxygenated and de-oxygenated blood, and the wavelength-dependent magnitude of light scattering in relationship to absorbance, according to the invention. The base absorbances, which are employed as inputs to the oximetry model of the invention, are fully adaptable to actual values of the tissue site chosen for actual pulse oximetry. In one embodiment of the invention, the base absorbances range from 0.1-1 times the total combined OD equivalent of absorbance and scattering, which are also referred to DC values for being the components that are considered invariable by the blood pressure pulse.

For optimal signal-to-noise conditions, it is preferred that a site and tissue thickness be chosen that results in a combined 0.5-1.5 OD for total blood absorbances and scattering intensity. For extreme, typical in-vitro applications, such as pulsed, lyzed blood, for example, the scattering term can approach zero.

FIG. 3 represents initial oxygenation conditions, wherein the O₂ saturation of arterial blood is at 100% and the venous return blood is at 60% O₂ saturation. Compared to published oxyHb and deoxyHb spectra data, it is surprising that there is a substantially larger oxyHb content in the combined total “DC” or the OD of all blood under interrogation. It is important to understand how this venous saturation affects the overall spectral absorbances, which is best exemplified in the shift of the isosbestic point to lower wavelengths.

In addition to the oximetry model of the invention having adjustable DC parameters and values, there are the corresponding pulse time-variable components and parameters, also referred to as AC. AC variables are referred to as such for being the components that are considered variable by the blood pressure pulse.

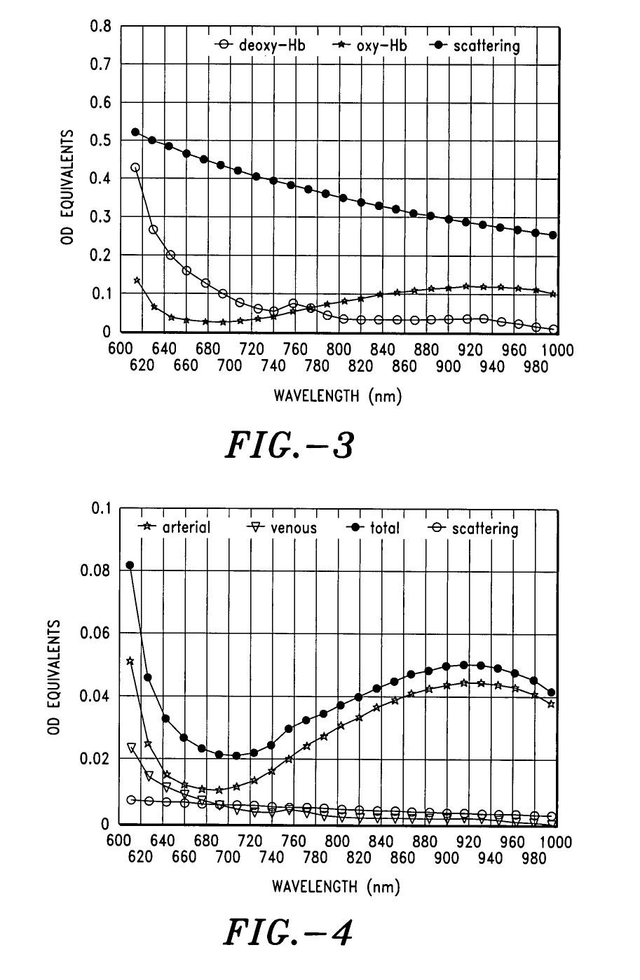

Referring now to FIG. 4, there is shown a graphical illustration of OD spectra at relevant wavelengths for oximetry of variable absorbances (AC), an example at one condition of oxygenated and de-oxygenated blood and the wavelength-dependent magnitude of light scattering in relationship to absorbance, according to the invention. The variable absorbances (AC), i.e. AC inputs to the oximetry model, are similarly fully adaptable to actual values of the tissue site chosen for actual pulse oximetry and, which, according to at least one embodiment, can range from approximately 0.01% to 25% of the total DC values. For optimal signal-to-noise conditions at the lower end of pulse amplitudes, e.g., at fractions of 1%, preferably, the range is approximately 0.1-10%.

While a range of approximately 0.1-10% is preferred, the actual amplitude range is typically defined by the patient condition and patient monitoring site.

The rationale for introducing separate and additional AC components for venous blood absorbance and light scattering changes is based on the pulse wave pressure induced transient distortion of the tissue, which, in turn, causes temporal changes in the fraction of scattered light. In addition to the propagating pressure wave causing absorbance changes of venules and capillaries, it is reasonable to assume that an overlap exists in small vessels between oxygen extraction and pressure reduction by restriction over distance as the blood flows from clearly arterial to clearly venous vessels.

A more complete and therefore more relevant model for prediction of the ratio of logarithm is thereby obtained. As a result of such valuable predictions, corrections can be applied in actual pulse oximetric calculation of real patient data until the results are indexed to defined norm conditions, such as, for example, 3% pulse amplitude at 4% venous and 6% scattering pulsation of total pulse amplitude.

The oximetry model of the invention is thus fully adaptable to accept any percentage of non-arterial components. Therefore, the preferred range for the model can range from zero to over 20% of each of the venous and scattering components.

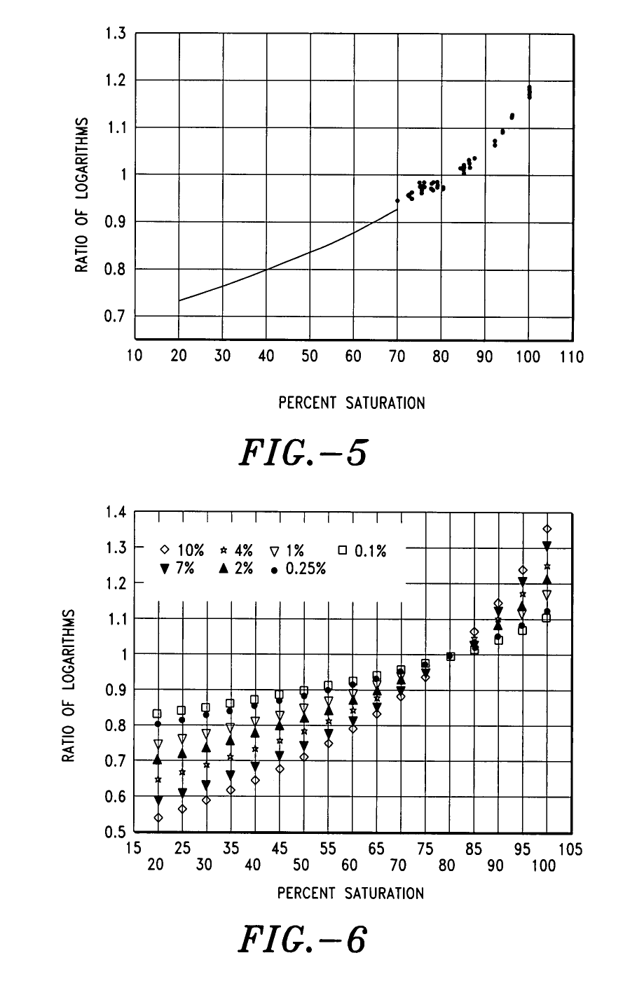

As is well known in the art, one of the most difficult areas of pulse oximetry is calibration below 70% O₂ saturation, which is inaccessible for ethical reasons. Referring to FIG. 5, there is shown a graphical illustration of the ratio of logarithms below 70% O₂ saturation based on a data fit of 70-100% O₂ saturation. As illustrated in FIG. 5, the oximetry model of the invention provides credible predictions of calibration data below 70% O₂ saturation.

According to the invention, the preferred range of predicted O₂ saturation is 50-70%. However, the entire range of 20-70% O₂ saturation is readily predictable.

An important feature of the model is accordingly the ability to predict O₂ saturation over the entire clinically expected range of conditions, including and most importantly, the expected observations at infrequently observed, extreme and, for ethical reasons, practically unattainable patient conditions.

### EXAMPLES

The following examples are provided to enable those skilled in the art to more clearly understand and practice the present invention. They should not be considered as limiting the scope of the invention, but merely as being illustrated as representative thereof.

### Example 1

Since pulse amplitude is highly variable between patients and can span more than two orders of magnitude from below 0.1% to over 25% O₂ saturation, it is desired (and, most times, required) that the variation in pulse amplitude be accounted for, i.e. proper corrections are employed.

In this example, the dependency of the ratio of logarithms over a range of O₂ saturations was determined for different total pulse amplitudes at a constant ratio of arterial to venous to scattering pulsation amplitudes of 8:1:1. Referring to FIG. 6, it can be seen that the magnitude of the error introduced by pulse amplitude is very small around 80% O₂ saturation. The magnitude of the error introduced by pulse amplitude is, however, significant at high and low saturations.

The error that is introduced by pulse amplitude poses a significant problem in clinical settings. As is known in the art, in a clinical setting where extremely low or high pulse amplitudes are often encountered, the error is frequently ignored with the justification that for “normal” pulse amplitudes of 1-5%, the magnitude of the error induced is similar to that exhibited from other error sources.

However, as reflected in FIG. 6, pulse amplitude error is substantially reduced at normal conditions by virtue of the predictive oximetry model and by implementing such calculated results, per the described methods of the invention, such as state of the art oximeter software algorithms.

### Example 2

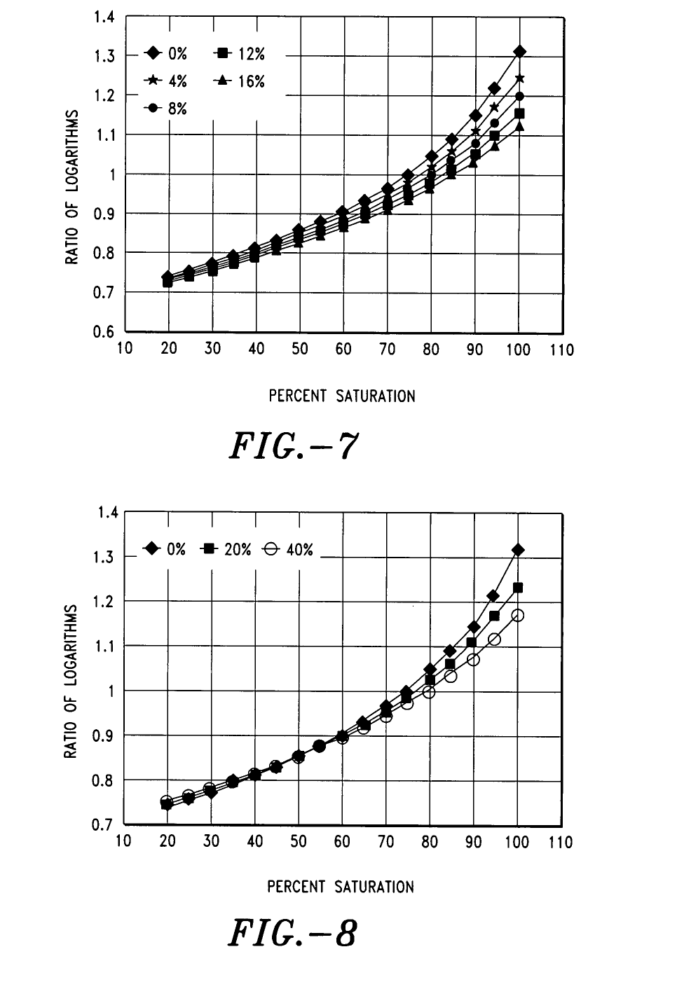

In the following example, the effect of venous pulsation on the ratio of logarithms and, hence, O₂ saturation determination was assessed. Referring now to FIG. 7, there is shown a graphical illustration of the effect of percent venous pulsatile component of total pulse amplitude on the ratio of logarithms over a range of O₂ saturations. As illustrated in FIG. 7, the effect of the venous pulsatile component on the ratio of logarithms is greater at the high end of the O₂ saturation range. It can also be seen that the sensitivity to O₂ saturation also decreases with increasing venous content.

Since a small and variable fraction of the total pulse amplitude is caused by pulsation of venous blood, the resulting ratio of logarithms is no longer a pure measurement of arterial saturation. Thus without correction, there are two sources of error, the percentage of venous pulsation and the effective saturation of such venous blood.

While it is difficult with conventional pulse oximetry methods to estimate the extent of venous pulsation, additional independent measurements, such as, for example, compression exsanguination at pressures exceeding venous pressure or alternatively, additional wavelengths that provide different absorbance coefficients for oxyHb and deoxyHb in combination with prediction by the described oximetry model can further enhance the effectiveness of the corrective means for the effect(s) of venous pulsation.

### Example 3

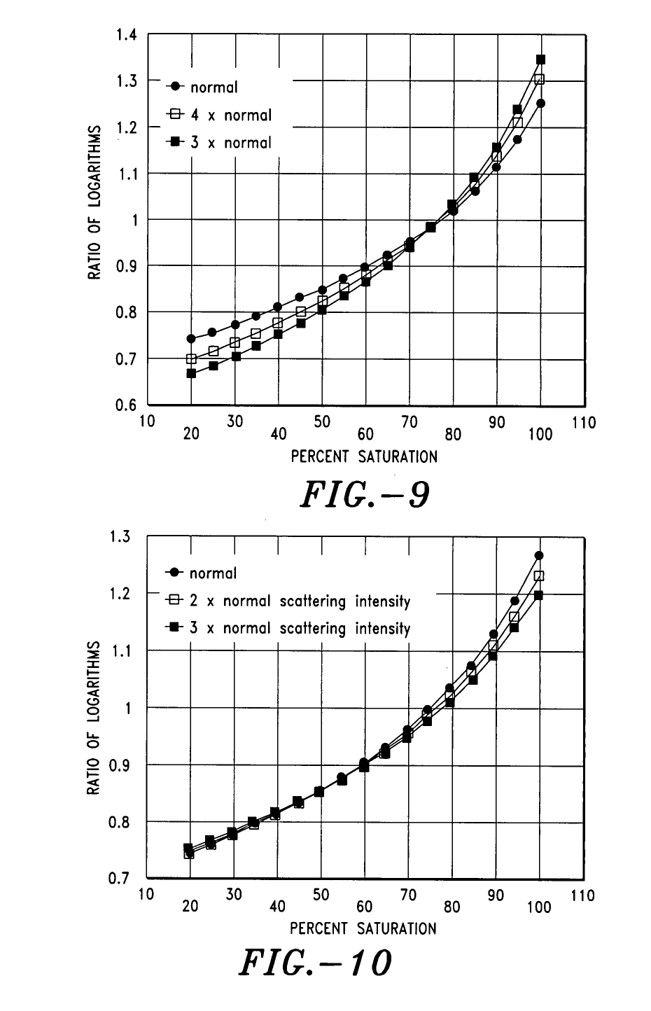

In the following example, the effect of scattering pulsation on the ratio of logarithms and, hence, O₂ saturation determination was assessed. Referring now to FIG. 8, there is shown a graphical illustration of the effect of percent non-optically absorbing pulsatile component of total pulse amplitude on the ratio of logarithms over a range of O₂ saturations. As illustrated in FIG. 8, the effect of scattering pulsations on the ratio of logarithms is similarly greater at the high end of the O₂ saturation range. The sensitivity to O₂ saturation also decreases with increasing scattering content.

While it may be difficult with conventional pulse oximetry methods to estimate the extent and effects of scattering intensity changes, as reflected in FIG. 8, the effects of scattering pulsation on the ratio of logarithms and, hence, O₂ saturation determination is minimal at normal conditions. Further, additional measurements during complete compression exanguinations (in combination with prediction by the described oximetry model) can further enhance the effectiveness of the corrective means for the effect(s) of scattering intensity changes.

### Example 4

In the following example, both absorbance (DC) and scattering (DC) were increased linearly by factors of two and three to assess the effects of tissue or appendage thickness on the ratio of logarithms. Non-linearities of the thickness function are to be expected, but as will be appreciated by one having ordinary skill in the art, the effect at different saturations is hard to predict without careful modeling or very extensive experimental work.

Referring now to FIG. 9, there is shown a graphical illustration of the effect of variations in relative finger thickness on the ratio of logarithms and, hence, O₂ saturation determination, when both absorbance and scattering are increased simultaneously and linearly. As illustrated in FIG. 9, the effect of variations in relative finger thickness on the ratio of logarithms is significantly greater at lower and higher O₂ saturation. However, the effects of finger thickness on the ratio of logarithms and, hence, O₂ saturation determination, is minimal at normal conditions, which is the standard for calibration of oximeters and sensors.

In the practical reality of pulse oximetry, the effective path length of a scattering medium is non-linear with finger thickness. Therefore, the predicted effects relate to the observable optical changes. The optical changes can, however, be empirically calibrated for variable finger thickness in a population of patients by virtue of the oximetry model and method of the invention. In addition, finger thickness can be measured, or alternatively, the DC values may be used as surrogates for finger thickness. In combination with this model, accurate corrections can be made and implemented in software of oximetry modules.

### Example 5

When an oximetry probe, which is calibrated to an average scattering medium, such as the index finger of a normal healthy person, is used on a similar size index finger, but of a dehydrated patient, for example, a change can be expected in scattering intensity due to the change in refractive index. In the following example, absorbance (DC) and scattering (DC) were similarly increased linearly by factors of two and three to assess the effects of variable scattering intensity of different tissue on the ratio of logarithms and, hence, O₂ saturation determination.

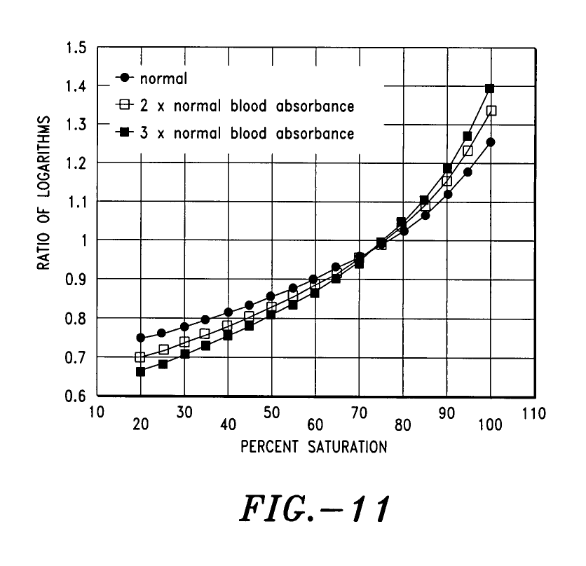

As illustrated in FIG. 10, it can be seen that the error introduced by scattering increased with increasing scattering at higher O₂ saturations. However, the error associated with scattering is minimal at normal conditions and below, i.e. <70% O₂ saturation. Additional measurements during complete compression exsanguintion, in combination with prediction by the described oximetry model, can further enhance the effectiveness of the corrective means.

### Example 6

In the following example, the effect of finger perfusion on the ratio of logarithms was assessed. Referring now to FIG. 11, there is shown a graphical illustration of the effect of variations in relative finger perfusion on the ratio of logarithms over a range of O₂ saturations.

As illustrated in FIG. 11, the effect of, i.e. error associated with, finger perfusion on the ratio of logarithms is similarly greater at the higher and lower O₂ saturation levels. However, as reflected in FIG. 11, the effect of finger perfusion on the ratio of logarithms and, hence, O₂ saturation determination, is minimal at normal conditions.

While it may be difficult with conventional pulse oximetry methods to estimate the extent of changes of tissue site perfusion by blood, particularly, in the arterioles and venules of the tissue under interrogation, additional independent measurements in combination with prediction by the described oximetry model can, and will, enhance the effectiveness of the corrective means.

As will be appreciated by one having ordinary skill in the art, the oximetry model and method of the invention provide accurate and, hence, effective means for predicting ratios of logarithms as functions of pulse amplitude and saturations. Such predictions are useful for the purpose of indexing each ratio of logarithms to a nearest tabulated value, whereby, a pulse amplitude corrected ratio of logarithms is obtained for a more accurate determination of oxygen saturation. Another example of an important utility of the oximetry model and method of the invention is the prediction of the ratio of logarithms at low saturations, i.e. saturations below 70%, which are not accessible by experimentation on human subjects.

The oximetry model and method of the invention further provides effective means for predicting the effects of (i) venous pulsation, (ii) scattering pulsation, (iii) finger (or biological tissue) thickness changes, (iv) biological tissue density changes, and (v) tissue perfusion on O₂ saturation determination(s), and effective corrective means therefore.

A further example of applied use of the described oximetry model is the prediction of effects on calibration caused by the presence in the interrogated blood sample of one or more additional hemoglobin components, such as the inactive species CO-Hb and/or met-Hb, and/or sickle cell Hb. These Hb species by their different spectral absorbances alter the amount of light measured at the oximetry wavelengths.

In general, the presence of species other than oxyHb and deoxyHb result in a variable but predictable offset at most wavelengths. Their presence and effect is readily accounted for in the model by also introducing their spectral absorbance characteristics as described above. As a function of their relative amounts, saturation and other terms, such as pulse amplitude effects, can be predicted.

By adhering to the spectroscopic principle that any component can be quantitatively measured if a sufficiently high number of wavelengths is employed, where different species absorb differently, and the signal outputs are utilized in mathematical combinations, such as multivariate analysis, the quantity of any such additional Hb component can be quantitatively assessed. The predictions of the model will thus allow for the use of simpler algorithms and reduced hardware.

Without departing from the spirit and scope of this invention, one having ordinary skill in the art can make various changes and modifications to the invention to adapt it to various usages and conditions. As such, these changes and modifications are properly, equitably, and intended to be, within the full range of equivalence of the following claims.

1. A method of determining oxygen saturation in a subject, comprising the steps of:
compiling a data base of measured spectral data characterizing the transmission of light through a tissue of interest in the subject, said spectral data including pulsatile AC and non-pulsatile DC components of said transmitted light, and spectral values of oxyhemoglobin (oxyHb) and deoxyhemoglobin (deoxyHb);
determining light scattering intensity (I) of said AC and DC components;
determining absorbed pulsatile components and non-pulsatile components as a function of said oxyHb and deoxyHb values;
determining total pulsatile optical density as a function of said absorbed pulsatile components and said AC component scattered light intensity;
determining total non-pulsatile optical density as a function of said absorbed non-pulsatile components and said DC component scattered light intensity;
determining a mathematical relationship between at least one pulsatile AC parameter and at least one non-pulsatile DC parameter; and
estimating oxygen saturation based on said mathematical relationship.

2. The method of claim 1, wherein said pulsatile AC parameter comprises a total pulsatile transmittance equivalent value and said non-pulsatile DC parameter comprises a total non-pulsatile transmittance equivalent value.

3. The method of claim 2, including the steps of determining said total pulsatile transmittance equivalent value as a function of said total pulsatile optical density and determining said total non-pulsatile transmittance equivalent value as a function of said total non-pulsatile optical density.

4. (canceled)

4. The method of claim 1, wherein said mathematical relationship comprises a ratio of logarithms (R), said ratio of logarithms (R) being determined according to the following equation
R=(log (T_{p@W1}/log T_{n,p@W1)})/log (T_{p@W2}/log T_{n,p @W2}))
wherein, Tₚ represents said total pulsatile transmittance equivalent value, T_{n,p} represents said total non-pulsatile transmittance equivalent value, W1 represents a first wavelength and W2 represents a second wavelength.

6-7. (canceled)

5. The method of claim 1, wherein said total pulsatile optical density (OD_{t,p}) is determined according to the following equation
OD_{t,p}=(a*OD_{a,p})+(b*OD_{v,p})+(c*OD_{o,p})+(d*I_{s,p})
wherein, a represents a fractional coefficient for arterialized, fully oxygenated pulsatile optical density, b represents a fractional coefficient for venous pulsatile optical density, c represents a fractional coefficient for pulsatile optical density of other hemoglobin (Hb) components, d represents a fractional coefficient for pulsatile scattering intensity, I_{s,p} represents pulsatile scattering intensity, OD_{a,p} represents arterialized, fully oxygenated pulsatile optical density, OD_{v,p} represents venous, fully oxygenated pulsatile optical density, and OD_{o,p} represents pulsatile optical density of other Hb components.

9-12. (canceled)

6. The method of claim 1, wherein said total non-pulsatile optical density (OD_{t,np}) is determined according to the following equation
OD_{t,np}=(e*OD_{a,np})+(f*OD_{v,np})+(g*OD_{o,np})+(h*I_{s,np})
wherein, e represents a fractional coefficient for arterialized, fully oxygenated non-pulsatile optical density, f represents a fractional coefficient for venous, fully oxygenated non-pulsatile optical density, g represents a fractional coefficient for non-pulsatile optical density of other hemoglobin (Hb) components, h represents a fractional coefficient for non-pulsatile scattering intensity, I_{s,np} represents non-pulsatile scattering intensity, OD_{a,np} represents arterialized, fully oxygenated non-pulsatile optical density, OD_{v,np} represents venous, fully oxygenated non-pulsatile optical density, and OD_{o,np} represents non-pulsatile optical density of other hemoglobin (Hb) components.

14-22. (canceled)

7. A method of determining oxygen saturation in a subject, comprising the steps of:
compiling a data base of measured spectral data characterizing the transmission of light through a tissue of interest in the subject, said spectral data including pulsatile AC and non-pulsatile DC components of said transmitted light, and spectral values of oxyhemoglobin (oxyHb) and deoxyhemoglobin (deoxyHb);
determining light scattering intensity (I) of said AC and DC components;
determining absorbed pulsatile components and non-pulsatile components as a function of said oxyHb and deoxyHb values;
determining total pulsatile optical density as a function of said absorbed pulsatile components and said AC component scattered light intensity;
determining total non-pulsatile optical density as a function of said absorbed non-pulsatile components and said DC component scattered light intensity;
determining at least one total pulsatile transmittance equivalent value as a function of said total pulsatile optical density;
determining at least one total non-pulsatile transmittance equivalent value as a function of said total non-pulsatile optical density;
determining a ratio of logarithms as a function of said pulsatile and non-pulsatile transmittance equivalent values; and
estimating oxygen saturation based on said ratio of logarithms.

8. The method of claim 7, wherein said light scattering intensity (I) of said AC and DC components is determined according to the following equation
I=s/(γ^{exp})
wherein, γ represents the wavelength and s represents an adjustment factor.

9. The method of claim 8, wherein said adjustment factor (s) is adjusted to provide between approximately 60% and 80% of the total DC component at a wavelength equal to approximately 805 nm when the total DC component reaches a total optical density level of approximately 1.5.

10. The method of claim 7, wherein said total pulsatile optical density (OD_{t,p}) is determined according to the following equation
OD_{t,p}=(a*OD_{a,p})+(b*OD_{v,p})+(c*OD_{o,p})+(d*I_{s,p})
wherein, a represents a fractional coefficient for arterialized, fully oxygenated pulsatile optical density, b represents a fractional coefficient for venous pulsatile optical density, c represents a fractional coefficient for pulsatile optical density of other hemoglobin (Hb) components, d represents a fractional coefficient for pulsatile scattering intensity, I_{s,p} represents pulsatile scattering intensity, OD_{a,p} represents arterialized, fully oxygenated pulsatile optical density, OD_{v,p} represents venous, fully oxygenated pulsatile optical density, and OD_{o,p} represents pulsatile optical density of other Hb components.

27-30. (canceled)

11. The method of claim 7, wherein said total non-pulsatile optical density (OD_{t,np}) is determined according to the following equation
OD_{t,np}=(e*OD_{a,np})+(f*OD_{v,np})+(g*OD_{o,np})+(h*I_{s,np})
wherein, e represents a fractional coefficient for arterialized, fully oxygenated non-pulsatile optical density, f represents a fractional coefficient for venous, fully oxygenated non-pulsatile optical density, g represents a fractional coefficient for non-pulsatile optical density of other hemoglobin (Hb) components, h represents a fractional coefficient for non-pulsatile scattering intensity, I_{s,np} represents non-pulsatile scattering intensity, OD_{a,np} represents arterialized, fully oxygenated non-pulsatile optical density, OD_{v,np} represents venous, fully oxygenated non-pulsatile optical density, and OD_{o,np} represents non-pulsatile optical density of other hemoglobin (Hb) components.

32-36. (canceled)

12. The method of claim 7, wherein said total pulsatile transmittance equivalent value (Tₚ) is determined according to the following equation
Tₚ= 1/10^{ODt,p}

13. The method of claim 7, wherein said total non-pulsatile transmittance equivalent value (T_{n,p}) is determined according to the following equation
T_{n,p}= 1/10^{ODt,np}

14. The method of claim 7, wherein said ratio of logarithms (R) is determined according to the following equation
R=(log(T_{p@W1}/log T_{n,p@W1}))/log(T_{p@W2}/log T_{n,p@W2}))
wherein, Tₚ represents said total pulsatile transmittance equivalent value, T_{n,p} represents said total non-pulsatile transmittance equivalent value, W1 represents a first wavelength and W2 represents a second wavelength.

40-41. (canceled)

15. A method of determining oxygen saturation in a subject, comprising the steps of:
providing a pulse oximetry system, said pulse oximetry system including a tissue probe having a radiation emitter that is adapted to transmit light having a first wavelength through a tissue of interest and a radiation detector that is adapted to receive said transmitted light after transmission through said tissue;
measuring said transmission of light through said tissue of interest in the subject with said pulse oximetry system;
compiling a data base of measured spectral data characterizing said transmission of light through said tissue of interest, said spectral data including pulsatile AC and non-pulsatile DC components of said transmitted light, and spectral values of oxyhemoglobin (oxyHb) and deoxyhemoglobin (deoxyHb);
determining light scattering intensity of said AC and DC components;
determining absorbed pulsatile components and non-pulsatile components as a function of said oxyHb and deoxyHb values;
determining total pulsatile optical density as a function of said absorbed pulsatile components and said AC component scattered light intensity;
determining total non-pulsatile optical density as a function of said absorbed non-pulsatile components and said DC component scattered light intensity;
determining at least one total pulsatile transmittance equivalent value as a function of said total pulsatile optical density;
determining at least one total non-pulsatile transmittance equivalent value as a function of said total non-pulsatile optical density;
determining a ratio of logarithms as a function of said pulsatile and non-pulsatile transmittance equivalent values; and
estimating oxygen saturation based on said ratio of logarithms.

16. The method of claim 15, wherein said light scattering intensity of said AC and DC components is determined according to the following equation
I=s/(λ^{exp})
wherein, λ represents the wavength and s represents an adjustment factor.

44-45. (canceled)

17. The method of claim 15, wherein said total pulsatile optical density (OD_{t,p}) is determined according to the following equation
OD_{t,p}=(a*OD_{a,p})+(b*OD_{v,p})+(c*OD_{o,p})+(d*I_{s,p})
wherein, a represents a fractional coefficient for arterialized, fully oxygenated pulsatile optical density, b represents a fractional coefficient for venous pulsatile optical density, c represents a fractional coefficient for pulsatile optical density of other hemoglobin (Hb) components, d represents a fractional coefficient for pulsatile scattering intensity, I_{s,p} represents pulsatile scattering intensity, OD_{a,p} represents arterialized, fully oxygenated pulsatile optical density, OD_{v,p} represents venous, fully oxygenated pulsatile optical density, and OD_{o,p} represents pulsatile optical density of other Hb components.

47-50. (canceled)

18. The method of claim 15, wherein said total non-pulsatile optical density (OD_{t,np}) is determined according to the following equation
OD_{t,np}=(e*OD_{a,np})+(f*OD_{v,np})+(g*OD_{o,np})+(h*I_{s,np})
wherein, e represents a fractional coefficient for arterialized, fully oxygenated non-pulsatile optical density, f represents a fractional coefficient for venous, fully oxygenated non-pulsatile optical density, g represents a fractional coefficient for non-pulsatile optical density of other hemoglobin (Hb) components, h represents a fractional coefficient for non-pulsatile scattering intensity, I_{s,np} represents non-pulsatile scattering intensity, OD_{a,np} represents arterialized, fully oxygenated non-pulsatile optical density, OD_{v,np} represents venous, fully oxygenated non-pulsatile optical density, and OD_{o,np} represents non-pulsatile optical density of other hemoglobin (Hb) components.

52-55. (canceled)

19. The method of claim 15, wherein said total pulsatile and non-pulsatile transmittance equivalent values are determined at a plurality of wavelengths.

20. The method of claim 15, wherein said total pulsatile transmittance equivalent value (Tₚ) is determined according to the following equation
Tₚ= 1/10^{ODt,p}

21. The method of claim 15, wherein said total non-pulsatile transmittance equivalent value (T_{n,p}) is determined according to the following equation
T_{n,p}= 1/10^{ODt,np}

22. The method of claim 15, wherein said ratio of logarithms (R) is determined according to the following equation
R=(log (T_{p@W1}/log T_{n,p@W1))/(log (T}_{p@W2}/log T_{n,p@W2}))
wherein, Tₚ represents said total pulsatile transmittance equivalent value, T_{n,p} represents said total non-pulsatile transmittance equivalent value, W1 represents a first wavelength and W2 represents a second wavelength.

60-61. (canceled)

23. A method of calibrating a pulse oximetery system, comprising the steps of:
providing a pulse oximetry system, said pulse oximetry system including a tissue probe having a radiation emitter that is adapted to transmit light having a first wavelength through a tissue of interest and a radiation detector that is adapted to receive said transmitted light after transmission through said tissue;
measuring said transmission of light through said tissue of interest in the subject with said pulse oximetry system;
compiling a data base of measured spectral data characterizing the transmission of light through a tissue of interest in the subject, said spectral data including pulsatile AC and non-pulsatile DC components of said transmitted light, and spectral values of oxyhemoglobin (oxyHb) and deoxyhemoglobin (deoxyHb);
determining light scattering intensity of said AC and DC components;
determining absorbed pulsatile components and non-pulsatile components as a function of said oxyHb and deoxyHb values;
determining a corrected ratio of logarithms based on said absorbed pulsatile and non-pulsatile components, and said AC and DC component scattered light intensities, said corrected ratio of logarithms representing oxygen saturation in a subject.
